(19) United States
(12) Patent Application Publication
(10) Pub. No.: US 2026/0060632 A1
(43) Pub. Date: Mar. 5, 2026

(54) MOTION CORRECTION WITH LOCALLY LINEAR EMBEDDING FOR ULTRAHIGH RESOLUTION COMPUTED TOMOGRAPHY

(71) Applicant: Rensselaer Polytechnic Institute, Troy, NY (US)

(72) Inventors: Ge Wang, Loudonville, NY (US); Mengzhou LI, Troy, NY (US)

(73) Assignee: Rensselaer Polytechnic Institute, Tory, NY (US)

(21) Appl. No.: 19/104,453

(22) PCT Filed: Aug. 21, 2023
(86) PCT No.: PCT/US2023/030668

§ 371 Date: Feb. 18, 2025

Related U.S. Application Data
(60) Provisional application No. 63/399,346, filed on Aug. 19, 2022.
(60) Provisional application No. 63/524,955, filed on Jul. 5, 2023.

(51) Int. Cl. A61B 6/00; A61B 6/03; G06T 5/73; G06T 7/70; G06T 11/00

(52) U.S. Cl. CPC A61B 6/5264; A61B 6/035; A61B 6/4452; A61B 6/4458; A61B 6/5205; G06T 5/73; G06T 7/70; G06T 12/00; G06T 2207/10081; G06T 2207/10116; G06T 2207/20016; G06T 2207/30004; G06T 2210/41; G06T 2211/441

(57) ABSTRACT

A CT apparatus in which the x-ray source is coupled to a source robotic arm and the detector is coupled to a detector robotic arm. A motion correction module utilizes a locally linear embedding motion correction algorithm to estimate the geometry-describing parameters associated with the positions of the source and detector and the angle of the detector. These estimates are used to reconstruct the image data and produce corrected images with fewer errors resulting from patient movement, misalignments, and coordination issues in the system.

### CROSS REFERENCE TO RELATED APPLICATION(S)

This application claims the benefit of U.S. Provisional Application No. 63/399,346, filed Aug. 19, 2022, PCT Application No. PCT/US2023/016359 filed Mar. 27, 2023, and U.S. Provisional Application No. 63/524,955 filed Jul. 5, 2023, all of which are incorporated by reference as if disclosed herein in their entireties.

### GOVERNMENT LICENSE RIGHTS

This invention was made with government support under award number CA237267 and EB026646, both awarded by the National Institutes of Health. The government has certain rights in the invention.

### FIELD

The present disclosure relates to motion correction with locally linear embedding, and more particularly, to motion correction with locally linear embedding for photon-counting computed tomography (CT) utilizing robotic arms.

### BACKGROUND

Robotic-arm-based clinical micro-CT can provide significant benefits in many clinical applications. Such systems can provide 50 μm resolution and can minimize radiation dose by focusing on a volume of interest (VOI). The use of robotic arms provides more flexibility and other benefits associated with advanced non-circular trajectories, including: increased field of view, less scan time, and reduced metal artifacts and radiation dose. In systems with twin robots, i.e., a robot for the x-ray source and a robot for the x-ray detector, further agility is helpful in VOI localization with a limited size photon-counting detector.

Such systems do present some challenges, however. As one example, mechanical coordination error, in the form of, for example, system vibration, gravity-induced strain, thermal drift, backlash, system misalignment, coordination errors, and patient movement, can lead to image blurring or distortion if not appropriately corrected. Patient movement can be especially problematic in ultrahigh resolution clinical imaging.

As another example, interior tomography, such as is often employed for oral and maxillofacial cone-beam CT commonly collected laterally truncated projections. This data truncation invalidates most data consistency methods and also brings challenges to tomographic consistency-based motion estimation methods due to the truncation artifacts in the reconstruction.

### SUMMARY

According to a first embodiment, a computed tomography (CT) apparatus is provided that includes an x-ray source coupled to a source robotic arm, an x-ray detector coupled to a detector robotic arm and adapted to output scan data of a volume of interest (VOI) contained in an imaging object, and a computing device. The computing device comprises a motion correction module that includes software instructions for: estimating a set of geometry-describing parameters comprising the position of the x-ray source, the position of the x-ray detector, and the angular orientation of the x-ray detector based on the scan data and utilizing a locally linear embedding (LLE) motion correction algorithm; generating, via a reconstruction module, reconstructed image data from the estimated geometry-describing parameters; and outputting corrected image data based, at least in part, on the reconstructed image data, to form a corrected image of the VOI.

According to a second embodiment, a non-transitory computer-readable medium storing instructions for causing a computing device to perform at least the following steps is provided: estimate a set of geometry-describing parameters comprising the position of an x-ray source coupled to a source robotic arm, the position of an x-ray detector coupled to a detector robotic arm, and the angular orientation of the x-ray detector based on scan data received from the detector for a volume of interest (VOI) contained in an imaging object and utilizing a locally linear embedding (LLE) motion correction algorithm; generate, via a reconstruction module, reconstructed image data from the estimated geometry-describing parameters; and output corrected image data based, at least in part, on the reconstructed image data to form a corrected image of the VOI.

In some embodiments, there are nine geometry-describing parameters per view: the position of the x-ray source is defined by three source coordinates in three-dimensional space (stₓ, st_{z}, st_{y}), the Position of the x-Ray Detector is Defined by Three Detector Coordinates in three-dimensional space (dtₓ, dt_{z}, dt_{y}), and the angular position of the x-ray detector is defined by three rotation angles (θₓ, θ_{y}, θ_{z}), each rotation angle relative to a respective axis of the three-dimensional space.

In some embodiments, the LLE motion correction algorithm comprises software instructions for estimating each of the nine geometry-describing parameters by performing the following steps for each parameter: generating a sampling grid for the parameter; calculating forward projections corresponding to the samples on the sampling grid; finding the K projections on the projection grid for each scan data point associated with the sampling grid that are the nearest neighbors to the scan data point; optimizing the weights for the K neighbors; and updating the estimated parameter and image reconstruction. These steps are iterated until a convergence is reached for each parameter or terminated early for a specified number of iterations.

In some embodiments, the source robotic arm and the detector robotic arm are configured to perform a scan along a task specific scan trajectory.

In some embodiments, the corrected image has a resolution of at least 50 micrometers (μm).

In some embodiments, the geometry-describing parameters are optimized in the sequence dtₓ, dt_{z}, dt_{y}, θₓ, θ_{y}, θ_{z}, stₓ, st_{z}, st_{y}.

In some embodiments, for each iteration a sampling space for the sampling grid is reduced while maintaining the same number of samples to generate a finer sample grid having improved searching accuracy.

According to a third embodiment, a method for correcting motion between an x-ray source coupled to a source robotic arm and an x-ray detector coupled to a detector robotic arm is provided, comprising the steps of: estimating a set of geometry-describing parameters comprising the position of the x-ray source, the position of the x-ray detector, and the angular orientation of the x-ray detector based on scan data received from the detector for a volume of interest (VOI) contained in an imaging object and utilizing a locally linear embedding (LLE) motion correction algorithm; generating, via a reconstruction module, reconstructed image data from the estimated geometry-describing parameters; and outputting corrected image data based, at least in part, on the reconstructed image data, to form a corrected image of the VOI.

In some embodiments, the step of estimating further comprises the steps of: estimating each of the nine geometry-describing parameters by, for each parameter: generating a sampling grid for the parameter; calculating forward projections corresponding to the samples on the sampling grid; finding the K projections on the projection grid for each scan data point associated with the sampling grid that are the nearest neighbors to the scan data point; optimizing the weights for the K neighbors; and updating the estimated parameter and image reconstruction. These steps are iterated until a convergence is reached for each parameter or terminated early for a specified number of iterations.

In some embodiments, the method further comprises the step of performing a scan with the source robotic arm and detector robotic arm along an arbitrary scan trajectory. In some embodiments, the geometry-describing parameters are optimized in the sequence dtₓ, dt_{z}, dt_{y}, θₓ, θ_{y}, θ_{z}, stₓ, st_{z}, st_{y}. In some embodiments, the method further comprises the step of reducing a sampling space for the sampling grid for each iteration while maintaining the same number of samples to generate a finer sample grid having improved searching accuracy.

### BRIEF DESCRIPTION OF DRAWINGS

The drawings show embodiments of the disclosed subject matter for the purpose of illustrating features and advantages of the disclosed subject matter. However, it should be understood that the present application is not limited to the precise arrangements and instrumentalities shown in the drawings, wherein:

FIG. 1 is a functional block diagram of a micro-CT apparatus according to an embodiment of the present technology.

FIG. 2(a) is a flow chart of operations for a method for correcting motion between an x-ray source and an x-ray detector according to an embodiment of the present technology.

FIG. 2(b) is a flow chart showing additional detail of some of the operations in FIG. 2(a).

FIG. 3(a) is a schematic illustration of a traditional gradient descent-based sequential updating.

FIG. 3(b) is a schematic illustration of LLE-based parallel grid searching via densely sampling.

FIG. 4(a) shows random translations and rotations introduced to the source and detector for numerical simulation.

FIGS. 4(b) and (c) show axial and coronary views of the reconstruction with correct geometry and without correction, respectively, as a result of numerical simulation.

FIG. 5 shows a 3D view, bird's-eye view, and side view, respectively, of a scanning trajectory used in an experimental data acquisition on a sacrificed mouse.

FIG. 6(a) shows a plot of the root mean squared error (RMSE) of each reconstruction update in the projection domain during the correction iterations, demonstrating the convergence after 3 iterations for this embodiment of a numerical simulation.

FIG. 6(b) shows the image difference against the reference at the same axial slice displayed in a narrow window for the numerical simulation.

FIG. 7 shows additional comparison images before and after correction against the reference.

FIG. 8 shows a plot of the RMSE curve in the projection domain during reconstruction for an experimental demonstration of an embodiment.

FIG. 9 shows images reconstructed before and after correction for the experimental demonstration. (a), (b), and (c) are axial, sagittal, and coronary views of the corrected reconstruction; (e), (g), and (i) correspond to the corrected reconstruction while (f), (h), and (j) correspond to the uncorrected reconstruction. (e) and (f), (g) and (h), and (i) and (j) display the zoom-in regions of (a), (b), and (c), respectively.

Although the following Detailed Description will proceed with reference being made to illustrative embodiments, many alternatives, modifications, and variations thereof will be apparent to those skilled in the art.

### DETAILED DESCRIPTION

Generally, this disclosure relates to a robotic arm-based clinical micro-CT (CMCT) apparatus, method and/or system. It should be noted that a robotic arm-based clinical micro-CT apparatus, system and method, according to the present disclosure, may be utilized for numerous clinical imaging applications, whether or not explicitly mentioned herein, within the scope of the present disclosure.

As used herein, medical CT corresponds to CT with a spatial resolution on the order of tenths of millimeters (mm). In one nonlimiting example, a medical CT image may have spatial resolution of 0.3 mm. The terms “medical CT”, “clinical CT” (without micro-) and/or “conventional CT” are used interchangeably. As used herein, “micro-CT” corresponds to CT with spatial resolution of the order of tens of micrometers (μm). As used herein, a “clinical micro-CT” corresponds to a micro-CT configured to image regions of a human body. In one nonlimiting example, a clinical micro-CT image may have a spatial resolution of 50 μm.

A robotic arm-based X-ray imaging system is configured with an X-ray source coupled to a first (“source”) robotic arm and an X-ray detector coupled to a second (“detector”) robotic arm in some embodiments. In some embodiments, the X-ray source includes, but is not limited to, a micro-focus X-ray tube, a dual energy CT source, e.g., a single-source X-ray source configured to emit an X-ray beam in two energy spectra, or a single energy spectrum source. In some embodiments, the X-ray detector includes, but is not limited to, an energy-integrating detector (EID), a current integrating detector (CID), a dual-layer detector, and a photon-counting detector (PCD). In one nonlimiting example, each robotic arm is configured with six degrees of freedom. Some embodiments of a robotic-arm-based X-ray imaging system, according to the present disclosure, provide flexibility in scanning and may facilitate a variety of scanning trajectories. The scanning trajectories can be task-specific and may include relatively diverse tasks targeting various organs and locations.

X-ray photon-counting detectors (PCDs) may enable relatively high-resolution (HR) and low-noise imaging, thus providing a spectral dimension to raw data and a corresponding improvement in CT performance. Different from an energy-integrating detector (EID), PCD works in a pulse-counting mode and directly converts individual X-ray photons into corresponding charge signals which are then sorted into different energy bins based on respective pulse heights. Thus, an intensity and wavelength information of incoming photons may be simultaneously obtained. Advantageously, PCDs generally do not suffer from electronic noise effects and may provide a relatively small effective pixel size; e.g., around 0.11 mm×0.11 mm and 0.055 mm×0.055 mm. As used herein, “around” and “approximately” correspond to within ±10 percent (%). PCDs allow applying selected weights to polychromatic photons for improved contrast and dose efficiency. Additionally or alternatively, an energy discrimination ability of PCDs may help reduce beam hardening and metal artifacts, and/or may enable K-edge imaging and material decomposition.

Micro-focus X-ray tubes are used with PCDs in micro-CT applications, according to some embodiments of the present disclosure. In one nonlimiting example, a microfocus X-ray tube includes electron emitting and receiving constructs (i.e., portions). The X-ray tube is configured to emit X-ray photons. The receiving portion contains an anode with a photoconductor. In some embodiments, the emission portion contains a backplate, a substrate, a cathode, a gate electrode, and an array of field emission electron sources. In another example, a microstructured array anode target (MAAT) X-ray source is configured to provide a relatively higher flux than an ordinary X-ray source in phase contrast imaging applications. It is contemplated that such technologies could be combined into a micro-focus X-ray tube for temporal bone CT imaging; for example, with a focal spot size of about 0.1 mm or less, corresponding to a PCD element size.

Generally, this disclosure relates to a robotic arm-based clinical micro-CT apparatus, method and/or system. The robotic arm-based clinical micro-CT apparatus, method and/or system, according to the present disclosure, may include a clinical micro-CT scanner. In an embodiment, the robotic arm-based clinical micro-CT apparatus includes the clinical micro-CT scanner and a computing device. In one embodiment, the clinical micro-CT scanner includes a micro-focus tube, and a PCD, each mounted on a robotic arm. In some embodiments, the clinical micro-CT apparatus, e.g., clinical micro-CT scanner, is configured to perform interior tomography. In some embodiments, the clinical micro-CT apparatus, e.g., computing device, may be configured to implement deep learning.

This disclosure also relates to motion correction with locally linear embedding for photon-counting computed tomography (CT) in some embodiments. A method, apparatus, system, and/or computer-readable medium containing software instructions may be configured to reduce or eliminate image artifacts that results from rigid-structure motion of the subject being imaged.

X-ray photon-counting detector (PCD) offers low noise, high resolution, and spectral characterization, representing a next generation of CT and enabling new biomedical applications. It is well known that involuntary patient motion may induce image artifacts with conventional CT scanning, and this problem becomes more serious with PCD due to its high-resolution detector pitch and extended scan time. Embodiments of the present disclosure are directed to a locally linear embedding (LLE) correction method, particularly as applied to robotic arm-based scanning systems, which is especially desirable given the high cost of large-area PCD. Embodiments of the present disclosure also-perform incremental updating on gradually refined sampling grids for optimization of both accuracy and efficiency.

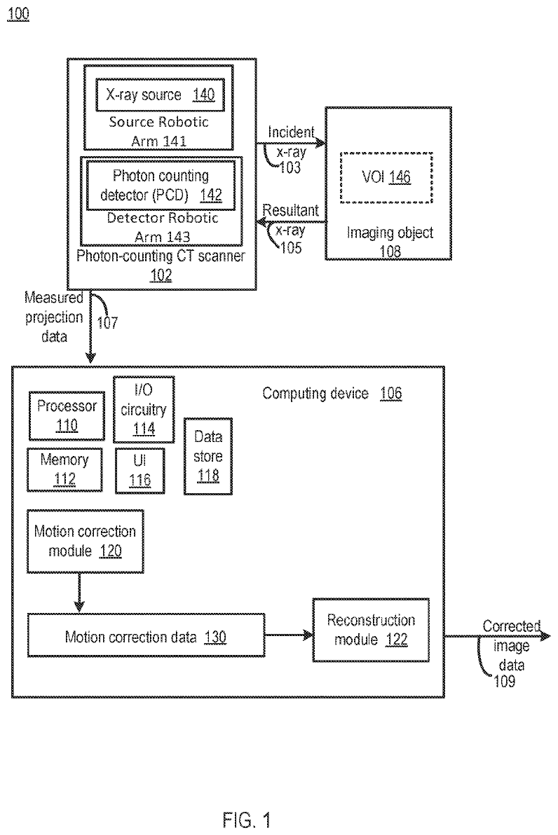

FIG. 1 illustrates a functional block diagram of an apparatus 100 that includes a motion correction system 101 for motion correction for photon-counting CT, according to several embodiments of the present disclosure. Apparatus 100 further includes a photon-counting CT scanner 102 for imaging an imaging object 108. Photon-counting CT scanner 102 includes an X-ray source 140 coupled to a source robotic arm 141 and a photon counting detector (PCD) 142 coupled to a detector robotic arm 143. In some embodiments, the robotic arms 141 and 143 each provide six degrees of freedom for positioning each of the X-ray source and the detector relative to one another in space. In other embodiments, fewer degrees of freedom are utilized for one or both of the robotic arms.

The imaging object 108 includes a volume of interest (VOI) 146, which is the apparatus's target for imaging. The VOI can be any suitable portion of a human or animal body, or any other object suitable for imaging via CT. Coupled to the CT scanner is computing device 106, which includes motion correction module 120. The motion correction module includes a motion correction algorithm configured to reduce or eliminate image artifacts that result from patient movement, system misalignment, coordination errors, etc.

Motion correction module 120 includes logic, in the form of software instructions in several embodiments, configured to receive the measured projection data (or scan data) 107 from the photon-counting CT scanner 102 and perform the LLE motion correction algorithm, as discussed above. In some embodiments, the LLE motion correction algorithm includes estimating a set of geometry-describing parameters comprising the position of the x-ray source, the position of the x-ray detector, and the angular orientation of the x-ray detector based on the scan data of the VOI. In some embodiments, because each of the robotic arms provides full and free movement of the source and detector in space, and because the detector can have an angular adjustment, the apparatus has nine degrees of freedom to be accounted for by the LLE motion correction algorithm. The nine geometry-describing parameters are: the position of the x-ray source is defined by three source coordinates in three-dimensional space (stₓ, st_{z}, st_{y}), the position of the x-ray detector is defined by three detector coordinates in three-dimensional space (dtₓ, dt_{z}, dt_{y}), and the angular position of the x-ray detector is defined by three rotation angles (θₓ, θ_{y}, θ_{z}), each rotation angle relative to a respective axis of the three-dimensional space.

In some embodiments, the LLE motion correction algorithm comprises software instructions for estimating each of the nine geometry-describing parameters by performing the following steps for each parameter: generating a sampling grid for the parameter; calculating forward projections corresponding to the samples on the sampling grid; finding the K projections on the projection grid for each scan data point associated with the sampling grid that are the nearest neighbors to the scan data point; optimizing the weights for the K neighbors; and updating the estimated parameter and image reconstruction. These steps are iterated until a convergence is reached for each parameter or terminated early for a specified number of iterations.

Reconstruction module 122 includes logic configured to receive the motion correction data 130 (including the geometry-describing parameters) outputted from the LLE motion correction algorithm and generate reconstructed image data, which is used to generate corrected image data 109 to form a corrected image of the VOI.

Computing device 106 may include, but is not limited to, a computing system (e.g., a server, a workstation computer, a desktop computer, a laptop computer, a tablet computer, an ultraportable computer, an ultramobile computer, a netbook computer and/or a subnotebook computer, etc.), and/or a smart phone. Computing device 106 includes a processor 110, a memory 112, input/output (I/O) circuitry 114, a user interface (UI) 116, and data store 118.

Processor 110 is configured to perform processing operations of apparatus 100. Memory 112 may be configured to store data associated with apparatus 104. I/O circuitry 114 may be configured to provide wired and/or wireless communication functionality for apparatus 100. For example, I/O circuitry 114 may be configured to receive measured correction data 107 and to provide corrected image data 109. UI 116 may include a user input device (e.g., keyboard, mouse, microphone, touch sensitive display, etc.) and/or a user output device, e.g., a display. Data store 118 may be configured to store one or more of measured correction data 107, corrected image data 109, and/or other data associated with any part of apparatus 100.

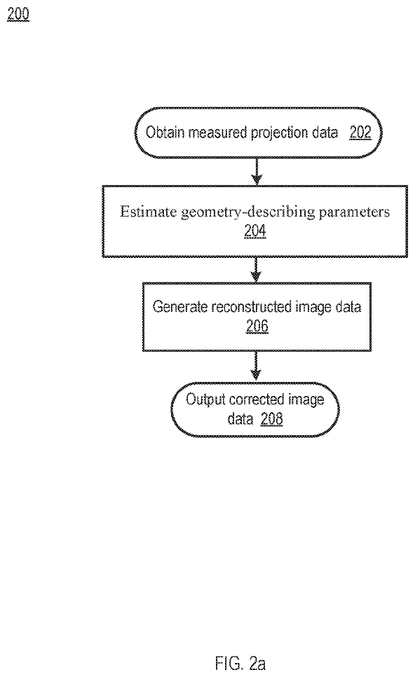

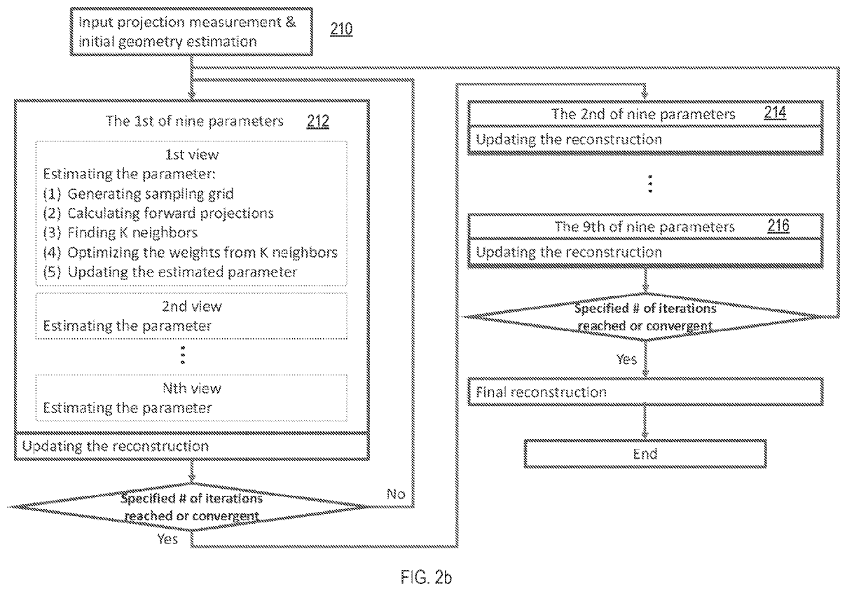

FIG. 2a is a flowchart 200 of operations for a method of motion correction for photon-counting CT, according to various embodiments of the present disclosure. In particular, the flowchart 200 illustrates a method for correcting motion between an x-ray source coupled to a source robotic arm and an x-ray detector coupled to a detector robotic arm. The operations may be performed, for example, by the system 100 of FIG. 1.

Operations of this embodiment begin with scanning a subject's VOI via a photon-counting CT scanner device to obtain measured projection data, or scan data, at operation 202. Operation 204 includes estimating a set of geometry-describing parameters associated with the source and detector from the measured projection data using a locally linear embedding (LLE) motion correction algorithm. Operation 206 includes generating, via reconstruction circuitry, reconstructed image data from the estimated geometry-describing parameters. Operation 208 includes outputting corrected image data based, at least in part, on the reconstructed image data to form a corrected image of the VOI. Although the operations of motion correction method are shown in flowchart 200 in a certain order, this is not meant to limit operations of the motion correction method to that specific order. For example, embodiments of the present disclosure may perform operations in any suitable order.

FIG. 2b is a flow chart showing details of the process for estimating the geometry-describing parameters and updating the reconstructions for each view to arrive at a final reconstruction according to an embodiment. To begin, the projection measurement and an initial geometry estimation is input at step 210. Then, the first of the nine geometry-describing parameters is estimated for each view by (1) generating a sample grid, (2) calculating forward projections, (3) finding K neighbors, (4) optimizing the weights from K neighbors, and (5) updating the estimated parameter at step 212. This process is undertaken for each of the N views and then the reconstruction is updated. In some embodiments, step 212 is repeated until convergence is reached or until a user specified number of iterations has been completed. The process of step 212 is then undertaken for each of the other nine geometry-describing parameters (214, 2016, . . . ) until convergence is reached for each parameter or a specified number of iterations is completed. Finally, the final reconstruction is made.

Thus, a method, according to the present disclosure, may be configured to reduce or eliminate image artifacts that results from rigid-structure motion of the subject being imaged by utilizing an LLE-based motion correction method for helical photon-counting CT, which decomposes the motion correction problem into each and every view with respective to individual parameters, and works iteratively in a highly parallel manner. Embodiments of the present disclosure exclude bad photon-counting detector pixels, and utilize unreliable volume masking, incremental updating, and incrementally refined gridding techniques synergistically. Thus, major improvements have been made in accuracy and efficiency of motion estimation and correction.

### Example: LLE-Based Motion Correction

A rigid movement (translation and rotation) of a patient can be equivalently viewed as the relative movement of the X-ray source and detector pair around a stationary patient. Hence, both patient movement, coordination errors, and system misalignment can be taken as a view-specific geometric calibration problem. The goal is to estimate the geometry that maximizes the consistency between the projection measurements and the reprojections of the associated reconstruction. Mathematically, this optimization problem is formulated as follows:

$${arg min}_{w , x} = {‖ b - A ( w ) x ‖}_{2}^{2} , \qquad ( 1 )$$

where b∈ [symbol] ^{HWN}^{ᵥ}^{×1} and x∈ [symbol] ^{(N\hat{ }3)×1} denote the measurements and a reconstructed volume, respectively, with [H,W] describing the size of each projection and Nᵥ representing the number of projections; A(w)∈ [symbol] ^{HWN}^{ᵥ}^{(N\hat{ }3)} is the system matrix corresponding to the geometry description w.

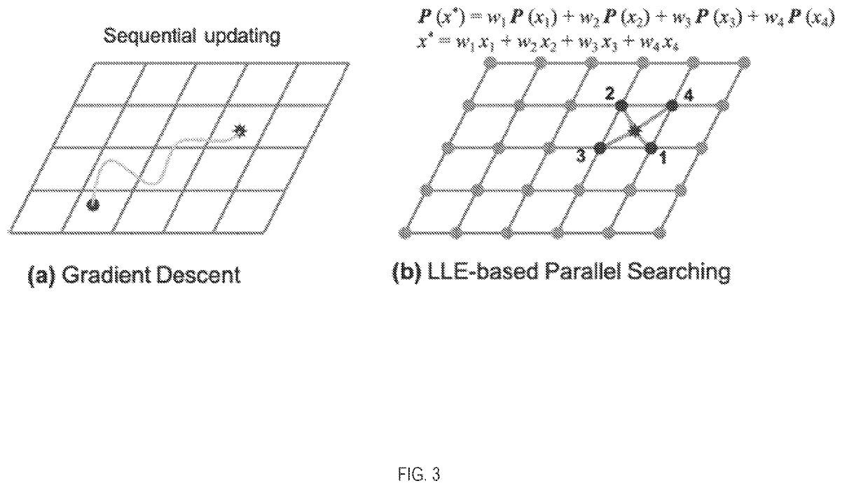

The optimization problem in Eq. 1 is often solved in an alternative updating fashion. That is, in some embodiments, first the reconstruction with a guessed w⁰ is performed to obtain x⁰, then refine the estimation with x⁰ and get w¹, improve the reconstruction with the refined w¹ and obtain x¹, so on and so forth, improving x and w in this loop. In some embodiments, SIRT (simultaneous iterative reconstruction technique) is used for reconstruction and an LLE-based method for geometry parameter estimation. Different from the sequential updating in gradient-based optimization, an LLE method favors parallel searching via densely grid sampling the parametric space. As illustrated in FIG. 3, if the sampling grid is dense enough, both the measurement and the corresponding parameter can be expressed as a linear combination of neighboring projections and samples, and a helpful property is that the two linear embeddings can share the same weights.

As mentioned above, a rigid movement (translation and rotation) of a patient can be viewed as the relative movement of the X-ray source and detector pair around a stationary patient. In fact, all three kinds of geometric errors (patient movement, geometric misalignment, and coordination errors) can be compensated for with the view-specific re-alignment of the source and the detector. Specifically for each view, the X-ray source position is described as S∈ [symbol] ³, the detector center position as D∈ [symbol] ³, and the detector row and column direction as u, v∈ [symbol] ² where [symbol] ² denotes the 3D unit sphere and u⊥v. In some embodiments, the target geometry is defined by {S₀, D₀, u₀, v₀}, and the deviation from the target is fully depicted with 3D translations of the S₀ and Do along three coordinate axes (stₓ, st_{y}, st_{z}, dtₓ, dt_{y}, dt_{z}) and 3D rotations of the u₀ and v₀ around the axes (θₓ, θ_{y}, θ_{z}). Thus, we have wᵢ=[stₓ, st_{y}, st_{z}, dtₓ, dt_{y}, dt_{z}, θₓ, θ_{y}, θ_{z}], where i=1, . . . , Nᵥ. The correction step for one view from the initial state is formulated as follows:

$$( \begin{matrix}D \\ 1\end{matrix} ) = {T}_{{dt}_{x}} {T}_{{dt}_{z}} {T}_{{dt}_{y}} ( \begin{matrix}{D}_{0} \\ 1\end{matrix} ) \cdot ( \begin{matrix}u & v \\ 1 & 1\end{matrix} ) = {R}_{{θ}_{x}} {R}_{{θ}_{y}} {R}_{{θ}_{z}} ( \begin{matrix}{u}_{0} & {v}_{0} \\ 1 & 1\end{matrix} ) \cdot ( \begin{matrix}S \\ 1\end{matrix} ) = {T}_{{st}_{x}} {T}_{{st}_{z}} {T}_{{st}_{y}} ( \begin{matrix}{S}_{0} \\ 1\end{matrix} ) , \qquad ( 2 )$$

where T_{(.)} and R_{(.)} are 4-by-4 translation and rotation matrices, respectively.

For estimation of wᵢ, a multidimensional dense grid sampling is too expensive in practice in some embodiments. Instead, in some embodiments, nine one-dimension searches are performed sequentially as a practical approximation. For each one-dimensional search, the following steps are used in some embodiments to refine the estimation:
1. Generate a sampling grid for the parameter and calculate the forward projections corresponding to the grid samples;
2. Find K projections on the projection grid that are most close to the measurement and embed the measurement with these K neighbors;
3. Estimate the parameter with the same embedding weights and corresponding K neighboring samples on the sample grid.

In some embodiments, the grid search for each degree of freedom can be implemented on GPU for acceleration in a view-independent manner. In some embodiments, the optimization sequence of dtₓ, dt_{z}, dt_{y}, θₓ, θ_{y}, θ_{z}, st_{y}, st_{z}, st_{y} is followed with a reconstruction update after each parameter update for all the views. The incremental updating strategy with coarse-to-fine sampling grids is also adopted for further acceleration and accuracy improvement as well as the bad pixel masking and unreliable volume masking techniques

### Numerical Simulation

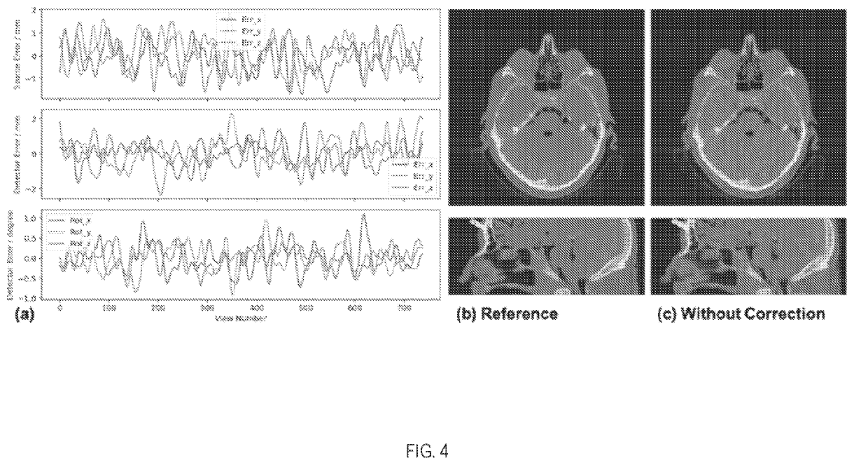

A portion of a human head image volume from a visible human project was used as a numerical phantom to validate this embodiment of the present technology. The volume was first re-sampled to have isotropic voxels of 0.53 mm³ resulting in a 512×512×200 volume after zero padding. A cone-beam scan consisted of 738 views was performed with a detector of 256×640 pixels and a pitch size of 1 mm. The source-to-isocenter distance and detector-to-isocenter distance were set to 625 mm and 500 mm, respectively. Random motions were added for the source and detector including translations and rotations as shown in FIG. 4(a). In this embodiment, smoothness of the motion was not assumed in order to mimic the random coordination errors of the robotic arms, working on the limit of the robot mechanical precision. A SIRT algorithm with 200 iterations was used to reconstruct the volume of 409×409×160 and 0.6253 mm³ voxel size for reconstruction updates. The images reconstructed with the correct trajectory (motion included) served as the ground truth as shown in FIG. 4(b), while the reconstruction without correction is in FIG. 4(c). Significant image blurring and artifacts can be observed in the images due to the misalignments. Color versions of the FIGS. 4-9 are available in the publication Mengzhou Li, Jana Bohacova, Josef Uher, Wenxiang Cong, Jay Rubinstein, and Ge Wang “Motion correction for robot-based x-ray photon-counting CT at ultrahigh resolution”, Proc. SPIE 12242, Developments in X-Ray Tomography XIV, 122420Y (14 Oct. 2022), the contents of which are incorporated by reference as if disclosed herein in its entirety.

For misalignment correction in this embodiment, the size of the sampling grid was set to 51 for all degrees of freedom, the translation sampling range to 12 detector pixels, and the rotation sampling range to 6 degrees. The correction cycle was iterated for 5 times with the sampling ranges shrunk by half after each iteration. The simulation was conducted in a noise-free situation. After the alignment, the images were reconstructed with 800 iterations of SIRT as the final result.

### Experimental Data Acquisition

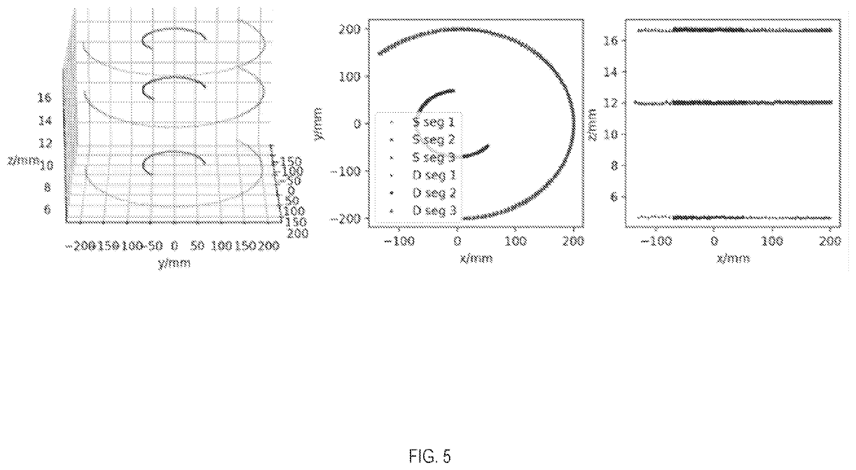

Robotic scans on a sacrificed mouse were also used to validate this embodiment. Three short scans were performed at different axial positions. The command trajectories of the source and detector are shown in FIG. 5. The source-to-detector distance and the source-to-isocenter distance were about 270 mm and 200 mm, respectively. Each arc had an angle about 222° for 111 projections. The detector consisted of 2×5 PCD chips with each containing 256×256 pixels, forming a frame size of 516×1290 with tiling gaps. The pitch was 55 μm.

For misalignment correction, the grid size was set to 51 for sampling, the translation range to 60 detector pixels, and the rotation range to 6 degrees. The number of iterations for correction was set to 5 with the sampling range shrunk by a factor of 0.7 per iteration. For computational acceleration, the image was reconstructed with a relatively large voxel size of 73 μm resulting a volume of 677×677×270, and the number of SIRT iterations was 200 for the initial 3 correction iterations then changed to 400 for the last 2 iterations. After correction, the volume was reconstructed with voxels of size 37 μm and 400 SIRT iterations for the final results, corresponding to a 1354×1354×541 volume.

### Results: Numerical Comparison

The simulation was conducted on a desktop computer equipped with a 24-core Intel 19-10920X CPU, 128 GB RAM and a NVIDIA RTX A5000. It took about 95 minutes to finish 5 iterations, in which reconstruction updates took about 50.4 minutes.

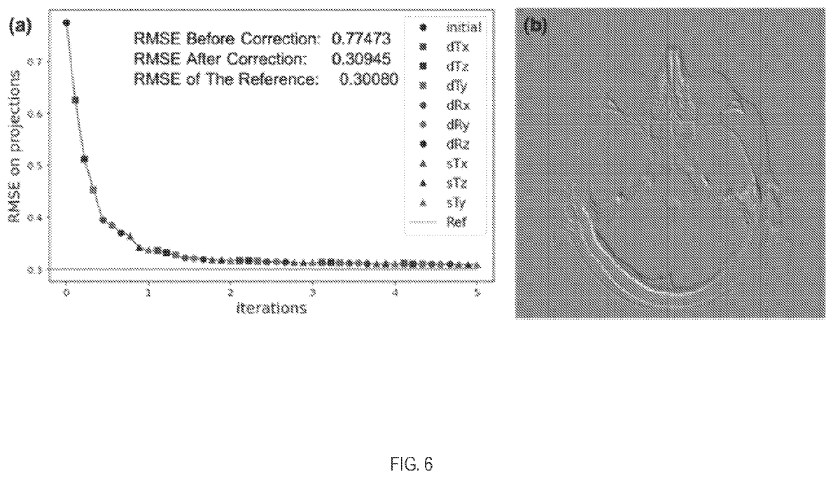

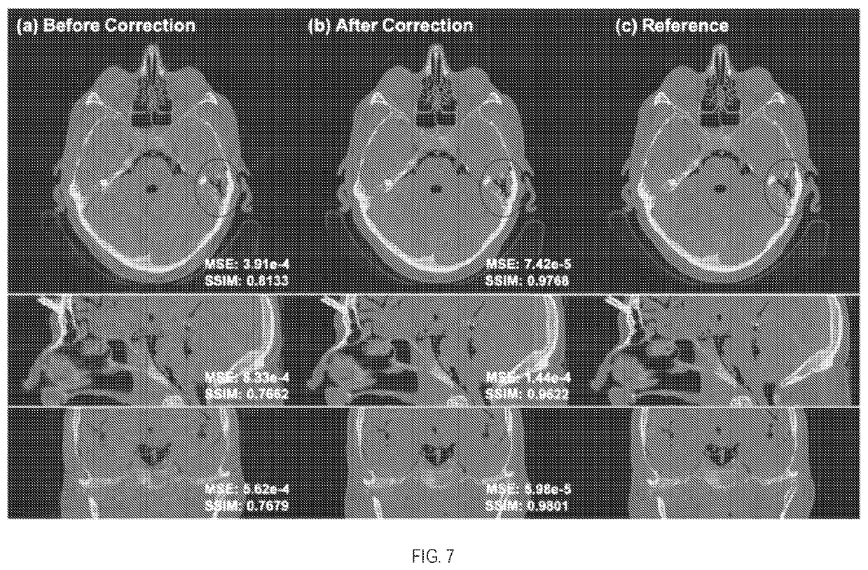

FIG. 6(a) shows the root mean squared error (RMSE) of each reconstruction update in the projection domain during the correction iterations, demonstrating the convergence after 3 iterations. The projection error was greatly reduced after correction, and the residual error caused by misalignment was less than 3% of the error caused by other factors (an insufficient number of SIRT iterations and increased voxel size of the reconstruction) as characterized by the projection error of the reference. FIG. 6(b) shows the image difference against the reference at the same axial slice displayed in a narrow window. Overall, the differences were less than 0.05. The enhanced edges along one diagonal direction suggest that there is a rotation between the reconstruction volume and the reference while the artifact fringes resulted from geometric misalignment became negligible.

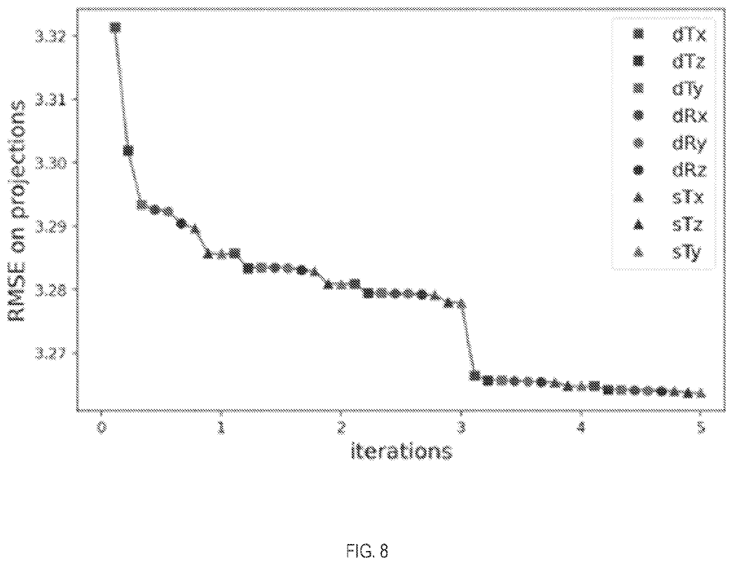

FIG. 7 displays more detailed comparison between the images before and after correction against the reference. Those images were reconstructed with 800 SIRT iterations. In the figure, misalignment artifacts were fully removed after the correction, and fine structures are revealed such as the bony structures of the inner ear in the circled region, and three white dots and one wide air slit in the gray matter indicated by the arrow. Despite a small misalignment, the corrected reconstruction agrees well quantitatively with the reference volume in terms of mean squared error (MSE) and Structure similarity index metric (SSIM), which demonstrates the effectiveness of this embodiment. Specifically, over 80% reduction in MSE and over 20% improvement in SSIM are obtained after correction.

### Results: Experimental Demonstration

The first 3 iterations took about 261 minutes (126 minutes for reconstruction updates) and the last 2 iterations took 258 minutes (168 minutes for reconstruction updates). As is seen, the reconstruction updates took over one half of the total time.

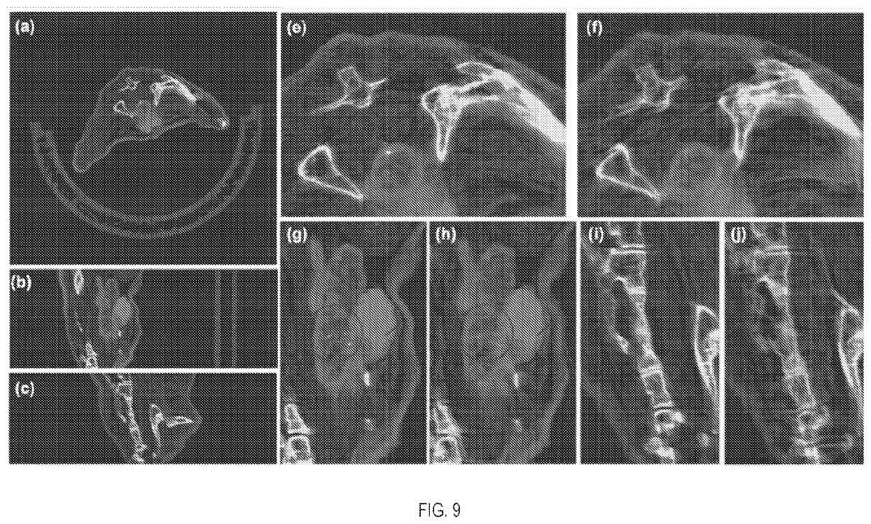

FIG. 8 shows the RMSE curve in the projection domain during the reconstruction. It converged in the first 3 iterations with 200 SIRT iterations for reconstruction updates, and by doubling the number of SIRT iterations, the RMSE was further decreased and converged to a smaller value in the last 2 iterations.

The final reconstruction of 37 μm voxels with 400 SIRT iterations are in FIG. 9. Compared to the reconstruction before correction, the corrected version is much sharper and with better contrast for bone structures and soft tissues boundaries. Inspecting zoom-in regions, with misalignment the bones appeared spiky and broken in FIG. 9(f) but the anatomical structures became more reasonable after correction in FIG. 9(e). The resolution also got enhanced as demonstrated by the improved boundary of white dots circled and the narrow slit pointed out by the upper arrow in FIGS. 9(g) and (h). FIG. 9(j) shows a significant number of artifacts induced by misalignment around the bone structures. Remarkably, after the correction in FIG. 9(i) the details were made much clearer in the absence of misalignment artifacts.

In this embodiment, a unified framework to incorporate all three types of geometric errors with nine degrees of freedom and LLE-based motion correction is presented. This embodiment does not involve fixing/making fiducials at high accuracy and is post-hoc relying only on the consistency between the re-projections and the measurements.

In this embodiment, the LLE method utilizes a grid searching strategy and thus is more computationally intensive than gradient-based sequential methods. Fortunately, due to its highly parallel feature it can be aided by a powerful GPU. Some embodiments, including this one, the geometry estimation is quite efficient (˜45 minutes) compared to a gradient-based method which takes about 1 hour 40 minutes with a volume of 256×256×120.

Deep learning methods or other techniques play a role in some embodiments for acceleration. Additionally, this embodiment delivers correct image reconstruction without misalignment artifacts as demonstrated in the simulation study and physical experiments,

In sum, this embodiment comprises a reference-free all-in-one geometric calibration and rigid motion correction method for robotic CT with an arbitrary scanning trajectory, which is based on the LLE idea combined with fine-to-coarse grid searching strategy. This embodiment is post-hoc and makes no assumptions about the smoothness of errors over viewing angulation. The effectiveness of this embodiment has been verified in the simulation study showing over 80% reduction in MSE and over 20% improvement in SSIM values, and in the experimental demonstrating sharper images with clearer anatomical details after correction. This and other embodiments are applicable to robotic CT imaging as well as conventional CT imaging for biomedical, industrial and other applications.

As used in any embodiment herein, the terms “logic” and/or “module” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

“Circuitry,” as used in any embodiment herein, may include, for example, singly or in any combination, hardwired circuitry, programmable circuitry such as computer processors comprising one or more individual instruction processing cores, state machine circuitry, and/or firmware that stores instructions executed by programmable circuitry. The logic and/or module may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc.

Memory 112 may include one or more of the following types of memory: semiconductor firmware memory, programmable memory, non-volatile memory, read only memory, electrically programmable memory, random access memory, flash memory, magnetic disk memory, and/or optical disk memory. Either additionally or alternatively system memory may include other and/or later-developed types of computer-readable memory.

Embodiments of the operations described herein may be implemented in a computer-readable storage device having stored thereon instructions that when executed by one or more processors perform the methods. The processor may include, for example, a processing unit and/or programmable circuitry. The storage device may include a machine readable storage device including any type of tangible, non-transitory storage device, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of storage devices suitable for storing electronic instructions.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various features, aspects, and embodiments have been described herein. The features, aspects, and embodiments are susceptible to combination with one another as well as to variation and modification, as will be understood by those having skill in the art. The present disclosure should, therefore, be considered to encompass such combinations, variations, and modifications.

What is claimed is:

1. A computed tomography (CT) apparatus, comprising:
an x-ray source coupled to a source robotic arm;
an x-ray detector coupled to a detector robotic arm and adapted to output scan data of a volume of interest (VOI) contained in an imaging object; and
a computing device, comprising:
a motion correction module, comprising software instructions for:
estimating a set of geometry-describing parameters comprising the position of the x-ray source, the position of the x-ray detector, and the angular orientation of the x-ray detector based on the scan data and utilizing a locally linear embedding (LLE) motion correction algorithm;
generating, via a reconstruction module, reconstructed image data from the estimated geometry-describing parameters; and
outputting corrected image data based, at least in part, on the reconstructed image data, to form a corrected image of the VOI.

2. The apparatus of claim 1, wherein there are nine geometry-describing parameters per view: the position of the x-ray source is defined by three source coordinates in three-dimensional space (stₓ, st_{z}, st_{y}), the position of the x-ray detector is defined by three detector coordinates in three-dimensional space (dtₓ, dt_{z}, dt_{y}), and the angular position of the x-ray detector is defined by three rotation angles (θₓ, θ_{y}, θ_{z}), each rotation angle relative to a respective axis of the three-dimensional space.

3. The apparatus of claim 2, wherein the LLE motion correction algorithm comprises software instructions for:
estimating each of the nine geometry-describing parameters by, for each parameter:
generating a sampling grid for the parameter;
calculating forward projections corresponding to the samples on the sampling grid;
finding the K projections on the projection grid for each scan data point associated with the sampling grid that are the nearest neighbors to the scan data point;
optimizing the weights for the K neighbors; and
updating the estimated parameter and image reconstruction; and
iterating the above steps until a convergence or a specified number of iterations is reached for each parameter.

4. The apparatus of claim 1, wherein the source robotic arm and the detector robotic arm are configured to perform a scan along a task specific scan trajectory.

5. The apparatus of claim 1, wherein the corrected image has a resolution of at least 50 micrometers (μm).

6. The apparatus of claim 3, wherein the geometry-describing parameters are optimized in the sequence dtₓ, dt_{z}, dt_{y}, θₓ, θ_{y}, θ_{z}, stₓ, st_{z}, st_{y}.

7. The apparatus of claim 3, wherein for each iteration a sampling space for the sampling grid is reduced while maintaining the same number of samples to generate a finer sample grid having improved searching accuracy.

8. A non-transitory computer-readable medium storing instructions for causing a computing device to:
estimate a set of geometry-describing parameters comprising the position of an x-ray source coupled to a source robotic arm, the position of an x-ray detector coupled to a detector robotic arm, and the angular orientation of the x-ray detector based on scan data received from the detector for a volume of interest (VOI) contained in an imaging object and utilizing a locally linear embedding (LLE) motion correction algorithm;
generate, via a reconstruction module, reconstructed image data from the estimated geometry-describing parameters; and
output corrected image data based, at least in part, on the reconstructed image data to form a corrected image of the VOI.

9. The medium of claim 8, wherein there are nine geometry-describing parameters per view: the position of the x-ray source is defined by three source coordinates in three-dimensional space (stₓ, st_{z}, st_{y}), the position of the x-ray detector is defined by three detector coordinates in three-dimensional space (dtₓ, dt_{z}, dt_{y}), and the angular position of the x-ray detector is defined by three rotation angles (θₓ, θ_{y}, θ_{z}), each rotation angle relative to a respective axis of the three-dimensional space.

10. The medium of claim 9, wherein the LLE motion correction algorithm comprises instructions to:
estimate each of the nine geometry-describing parameters by, for each parameter:
generating a sampling grid for the parameter;
calculating forward projections corresponding to the samples on the sampling grid;
finding the K projections on the projection grid for each scan data point associated with the sampling grid that are the nearest neighbors to the scan data point;
optimizing the weights for the K neighbors; and
updating the estimated parameter and image reconstruction; and
iterating the above steps until a convergence or a specified number of iterations is reached for each parameter.

11. The medium of claim 8, wherein the corrected image has a resolution of at least 50 micrometers (μm).

12. The medium of claim 10, wherein the geometry-describing parameters are optimized in the sequence div, dt_{z}, dt_{y}, θₓ, θ_{y}, θ_{z}, stₓ, st_{z}, st_{y}.

13. The medium of claim 10, wherein for each iteration a sampling space for the sampling grid is reduced while maintaining the same number of samples to generate a finer sample grid having improved searching accuracy.

14. A method for correcting motion between an x-ray source coupled to a source robotic arm and an x-ray detector coupled to a detector robotic arm, comprising the steps of:
estimating a set of geometry-describing parameters comprising the position of the x-ray source, the position of the x-ray detector, and the angular orientation of the x-ray detector based on scan data received from the detector for a volume of interest (VOI) contained in an imaging object and utilizing a locally linear embedding (LLE) motion correction algorithm;
generating, via a reconstruction module, reconstructed image data from the estimated geometry-describing parameters; and
outputting corrected image data based, at least in part, on the reconstructed image data, to form a corrected image of the VOI.

15. The method of claim 14, wherein there are nine geometry-describing parameters per view: the position of the x-ray source is defined by three source coordinates in three-dimensional space (stₓ, st_{z}, st_{y}), the position of the x-ray detector is defined by three detector coordinates in three-dimensional space (dtₓ, dt_{z}, dt_{y}), and the angular position of the x-ray detector is defined by three rotation angles (θₓ, θ_{y}, θ_{z}), each rotation angle relative to a respective axis of the three-dimensional space.

16. The method of claim 15, wherein the step of estimating further comprises estimating each of the nine geometry-describing parameters by, for each parameter:
generating a sampling grid for the parameter;
calculating forward projections corresponding to the samples on the sampling grid;
finding the K projections on the projection grid for each scan data point associated with the sampling grid that are the nearest neighbors to the scan data point;
optimizing the weights for the K neighbors; and
updating the estimated parameter and image reconstruction; and
iterating the above steps until a convergence or a specified number of iterations is reached for each parameter.

17. The method of claim 14, further comprising the step of performing a scan with the source robotic arm and detector robotic arm along an arbitrary scan trajectory.

18. The method of claim 16, wherein the geometry-describing parameters are optimized in the sequence dtₓ, dt_{z}, dt_{y}, θₓ, θ_{y}, θ_{z}, stₓ, st_{z}, st_{y}.

19. The method of claim 16, further comprising the step of reducing a sampling space for the sampling grid for each iteration while maintaining the same number of samples to generate a finer sample grid having improved searching accuracy.
